(19) United States
(12) Patent Application Publication
(10) Pub. No.: US 2005/0273048 A1
(43) Pub. Date: Dec. 8, 2005

(54) Needle-free single-use cartridge and injection system

(76) Inventor: Sergio Landau, Laguna Niguel, CA (US)

Correspondence Address: KOLISCH HARTWELL, P.C., 520 S.W. YAMHILL STREET, SUITE 200, PORTLAND, OR, 97204, US

(21) Appl. No.: 10/976,342

(22) Filed: Oct. 26, 2004

Related U.S. Application Data
(63) Continuation-in-part of application No. 10/861,891, filed on Jun. 4, 2004.

(51) Int. Cl. A61M 5/30

(52) U.S. Cl. 604/68

(57) ABSTRACT

A needle-free injection system, including a spring-powered injection device and an arming mechanism. The arming mechanism is configured to be operatively engaged with the injection device so that a spring of the injection device is mechanically coupled with the arming mechanism, the coupling of the spring and arming mechanism being such that operation of an actuator of the arming mechanism causes the spring of the injection device to be compressed, thereby arming the injection device. The arming mechanism may be further configured to automatically engage and release the mechanical coupling used to compress the spring.

### CROSS-REFERENCE TO RELATED APPLICATION

This application is a continuation-in-part of U.S. patent application Ser. No. 10/861,891, filed Jun. 4, 2004, which is hereby incorporated by reference in its entirety for all purposes.

### BACKGROUND

Needle-free injection systems provide an alternative to standard fluid delivery systems, which typically use a needle adapted to penetrate the outer surface of an injection site. Typically, needle-free injection systems are designed to eject the fluid from a fluid chamber with sufficient pressure to allow the fluid to penetrate the target to the desired degree. For example, common applications for needle-free injection systems include delivering intradermal, subcutaneous and intramuscular injections into or through a recipient's skin. For each of these applications, the fluid must be ejected from the system with sufficient pressure to allow the fluid to penetrate the tough exterior dermal layers of the recipient's skin.

There has been increased interest in using needle-free injection systems to deliver injections to large numbers of individuals, i.e. for inoculations, immunizations, etc. When using the same device to deliver inoculations, immunizations or the like, it is desirable for the device to be reloaded and capable of delivering the next injection relatively quickly, i.e., without significant time passing between injections. However, preventing cross-contamination between injection recipients must be a priority. Thus, it is desirable to provide a device that allows a user to move with reasonable speed from one injection recipient to another while maintaining adequate protections against cross-contamination. In addition, it will often be desirable to obtain the above advantages while also keeping waste to a minimum (e.g., by avoiding unnecessary disposal of portions of the injection system).

### BRIEF DESCRIPTION OF THE DRAWINGS

FIG. 1A is a view of a needle-free injection system according to the present description.

FIGS. 1B and 1C are views showing an alternate embodiment of a needle-free injection system according to the present description, including an injection device that may be operatively engaged with an arming mechanism.

FIG. 2 is a cross-sectional view of a nozzle assembly and ejector mechanism according to the present description, showing the components in an armed state prior to delivery of an injection.

FIG. 3 is a cross-sectional view of the nozzle assembly and ejector mechanism of FIG. 2, showing the components in a stored and/or discharged state.

FIG. 4 is an exploded isometric view of a nozzle/filling assembly and vial adapter according to the present description.

FIG. 5 is a cross-sectional view of the nozzle/filling assembly of FIG. 4.

FIG. 6 is a partial isometric view showing selective attachment of the nozzle/filling assembly of FIGS. 4 and 5 to the front end of the ejector mechanism of FIGS. 2 and 3.

FIG. 7 is an isometric view of a plunger coupling device according to the present description, which may be used to operatively engage the nozzle/filling assembly plunger with the ejector mechanism of FIGS. 2 and 3, so as to cause retraction and advancement of the plunger during arming and discharge of the injection system.

FIG. 8 is a partial cross-sectional view showing use of the plunger coupling device of FIG. 7 to couple the nozzle/filling assembly plunge with a cable piston of the ejector mechanism.

FIG. 9 is a cross-sectional view showing engagement of a vial adapter with the nozzle/filling assembly of FIGS. 4 and 5 to enable delivery of injectable fluid from an external supply into a fluid chamber of the nozzle/filling assembly.

FIGS. 10 and 11 are partial isometric depictions which respectively show the nozzle/filling assembly of FIGS. 4 and 5 before and after a filling adapter is broken off, the filling adapter being broken off after filling of the device but prior to delivery of an injection.

FIG. 12 is a cross-sectional view showing how the nozzle/filling assembly and vial adapter of FIGS. 4 and 5 prevent attempts to refill the nozzle/filling assembly after detachment of the filling adapter.

FIG. 13 is an isometric view depicting a portion of the filling adapter of FIGS. 4, 5 and 9.

FIG. 14 is an exploded isometric view showing alternate embodiments of a vial adapter and nozzle/filling assembly according to the present description.

FIG. 15 is a cross-sectional view depicting operative engagement of the vial adapter and nozzle/filling assembly of FIG. 14, so as to enable a dose of injectable fluid from an external supply (e.g., a vial) to be loaded into the injection device.

FIG. 16 depicts a non-compliant attempt to fill the nozzle/filling assembly of FIGS. 14 and 15 after detachment of the filling adapter.

FIG. 17 is a partial cross-sectional view depicting further alternate embodiments of a vial adapter and nozzle/filling assembly according to the present description.

FIGS. 18 is an exploded isometric view showing another alternate embodiment of a vial adapter according to the present description.

FIG. 19 depicts the via adapter of FIG. 18 operatively engaged with an alternate nozzle/filling assembly according to the present description.

FIG. 20 is a cross sectional view depicting various details of the injection device shown in FIGS. 1B and 1C.

FIGS. 21-23 are views depicting aspects of the arming mechanism of FIGS. 1B and 1C.

### DETAILED DESCRIPTION

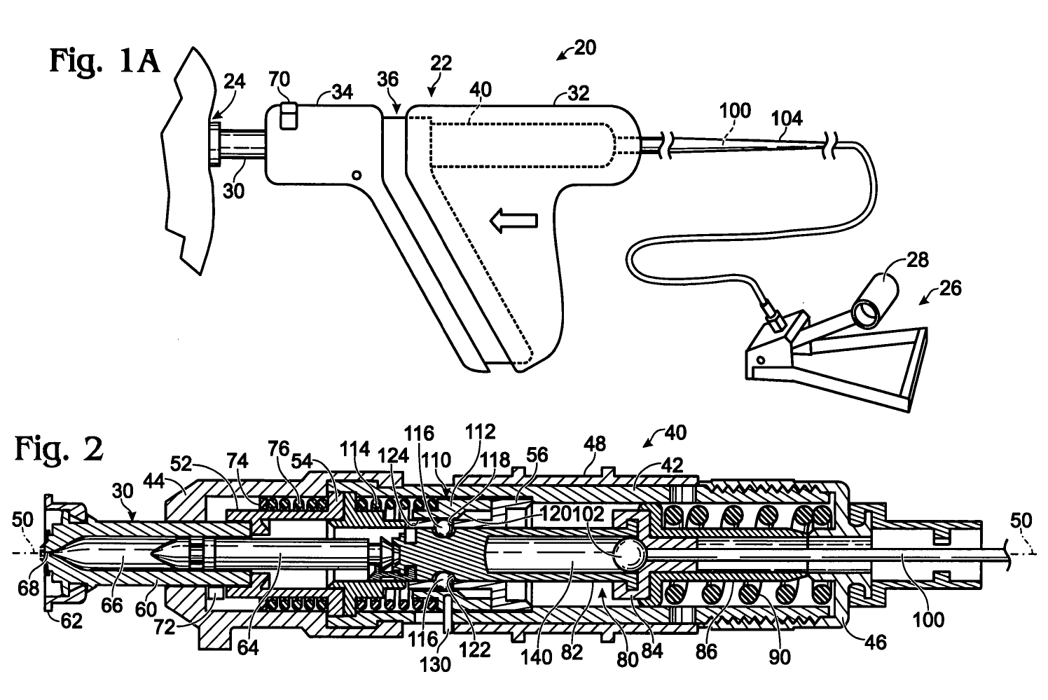

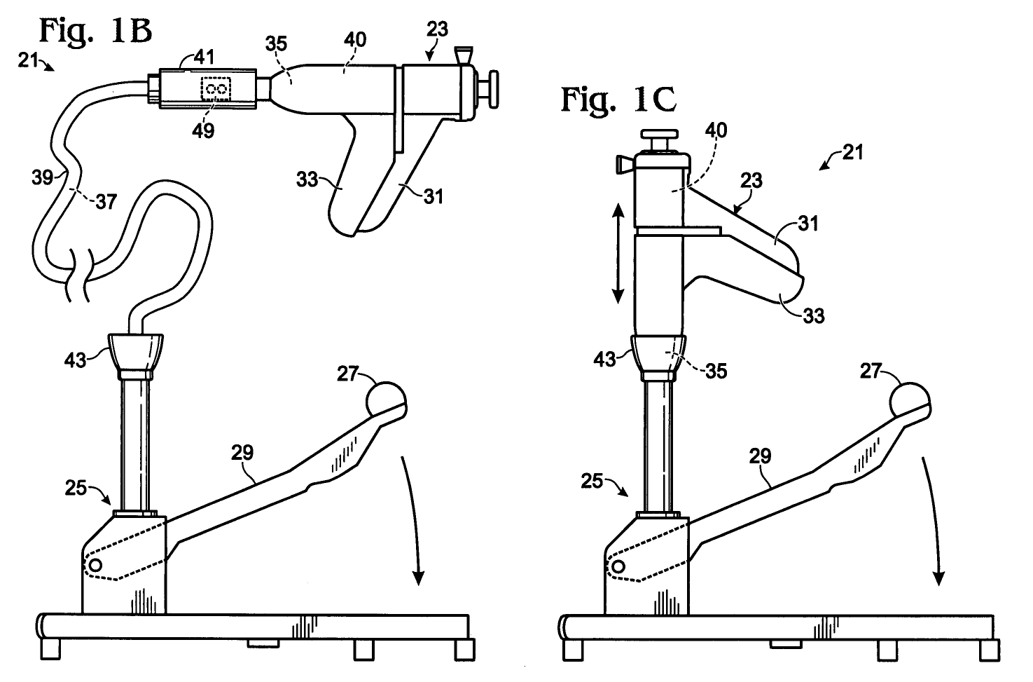

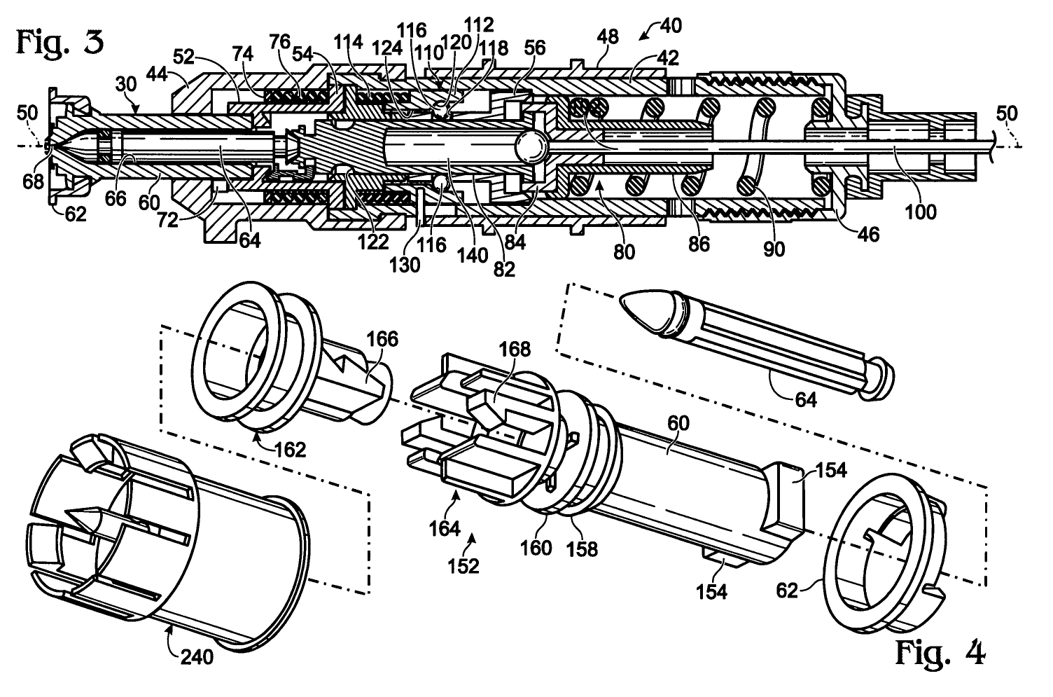

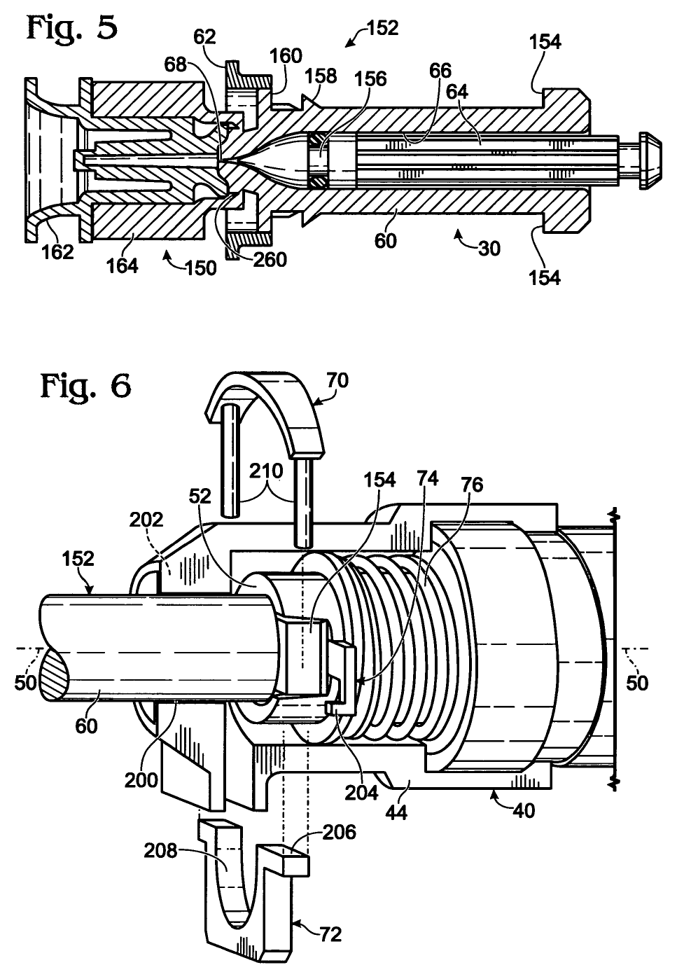

Referring to FIGS. 1A, 1B and 1C, various embodiments of a needle-free injection system 20 according to the present description are depicted. FIGS. 2 and 3 depict the structure and operation of a firing or ejector mechanism 40 that may be used in connection with the injection system to generate force for delivering pressurized injections of fluid, such as vaccines or other medications. FIGS. 4 and 5 depict a vial adapter 240, which connects to and seals an opening of a vial or other external supply of injectable fluid (not shown), and a nozzle/filling assembly 152.

As will be explained in more detail below, nozzle/filling assembly 152 typically is implemented as a single-use fluid cartridge that may be engaged with an ejector mechanism, such as that depicted in FIGS. 2 and 3. A fluid chamber within nozzle/filling assembly 152 may then be filled with a dose of injectable fluid. Typically, filling is accomplished from an external supply of fluid, which may include a vial adapter 240 that allows the external supply to be selectively coupled to the nozzle/filling assembly. After filling, the external supply of fluid is decoupled from the nozzle/filling assembly by simply removing the external supply and vial adapter 240 from engagement with the nozzle/filling assembly.

Regardless of the particular filling method used, the nozzle/filling assembly typically is configured so that, once it is filled, some user action is required before an injection can be delivered. Unless this enabling action is performed, the nozzle/filling assembly is incapable of effectively delivering an injection. Typically, the user action involves breaking a filling adapter 150 or other portion of the nozzle/filling assembly away from a remaining portion of the nozzle/filling assembly. Furthermore, in addition to allowing the injection to go forward, the user action (e.g., breaking off the filling adapter) disables the ability to refill the device. In the above example, filling adapter 150, mates with vial adapter 240, and thus enables nozzle/filling assembly 152 to be attached to the external supply of fluid. Accordingly, when the filling adapter is broken away, the nozzle/filling assembly can no longer be coupled with the vial of fluid, and thus cannot be refilled. The same act that allows the injection to go forward disables the ability of the device to be refilled. By simultaneously enabling the injection and disabling the ability to refill the nozzle/filling assembly, the user action ensures that the nozzle/filling assembly will not be re-used, thereby greatly reducing contamination risks. These and other features and advantages will be described in more detail below.

Referring now to FIG. 1, a first embodiment 20 of an injection system is depicted. As shown in the figure, system 20 may include an injection device 22 configured to deliver a pressurized injection of fluid to an injection site 24, and an actuating or arming mechanism 26, which may include a foot pedal 28. As described in more detail below, ejector mechanism 40 may be disposed within injection device 22, and may be armed via operation of arming mechanism 26. When discharged, ejector mechanism 40 causes pressurized fluid to be forcibly ejected from nozzle assembly 30 and into injection site 24. As explained below, in the depicted exemplary system, nozzle assembly 30 is the portion of nozzle/filling assembly 152 (FIGS. 4 and 5) that remains after filling adapter 150 is broken away to enable the injection to proceed.

Typically, some triggering operation is required to release ejector mechanism 40 from the armed state and deliver the injection. In the depicted example, injection device 22 includes an outer housing with two housing pieces 32 and 34 that are slidable relative to one another to trigger the injection. For example, rear housing piece 32 may be advanced relative to front housing piece 34 to close gap 36 and thereby trigger delivery of the injection.

FIGS. 1B and 1C depict an alternate embodiment 21 of an injection system according to the present description. System 21 includes an injection device 23 which may be repeatedly loaded or armed via operation of actuating or arming mechanism 25, which, similar to the embodiment of FIG. 1A, may include a foot or hand pedal 27 attached to a lever 29. Specifically, ejector mechanism 40 may be provided within a multiple piece housing (e.g., including front housing 31 and back housing 33), and may be armed operatively engaging the ejector mechanism with arming mechanism 25. As described in more detail below, ejector mechanism 40 typically includes a main spring that is compressed and then locked in a compressed state during arming of the device. The device is then discharged by allowing the spring to decompress.

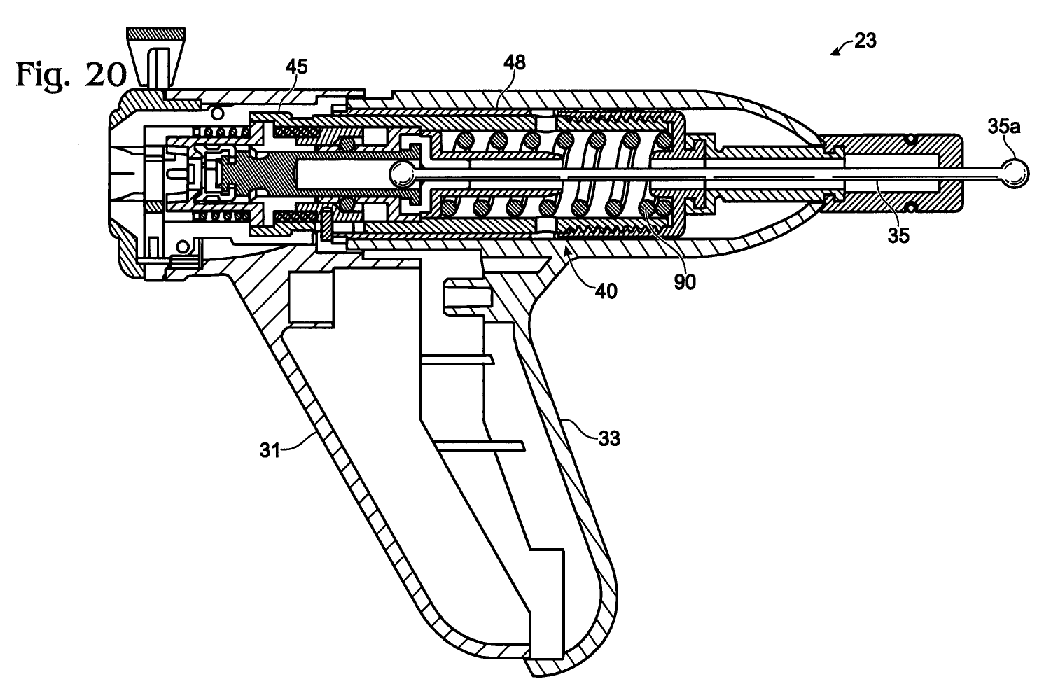

The main spring of ejector mechanism 40 typically is compressed by pulling or retracting a reciprocating member against the force of the ejector mechanism spring. This may be effected by pulling or urging against a connector secured to the reciprocating member. In the embodiments of FIGS. 1B and 1C, a short cable segment 35 is attached to ejector mechanism. Typically, as will be explained below with reference to FIG. 20, an end of cable segment 35 extends out from a rearward end of housing piece 33 and terminates in a ball or like anchor. In these embodiments, the exposed end of cable segment 35 may be pulled to retract internal mechanism(s) within injection device 23, so as to arm the system.

FIG. 1B depicts use of injection system 21 in a “tethered” mode, in which injection device 23 remains connected to arming mechanism 25 via an extension cable 37 disposed within cable housing 39. Extension cable 37 has a first end which is coupled via fitting 41 to the exposed end of short cable segment 35. The other end of extension cable 37 is operatively engaged with arming mechanism 25. To arm ejector mechanism 40, the user simply pushes pedal 27 downward (e.g., with their hand or foot). This results in pulling the cable segment 35 rearward relative to injection device 23, to thus compress the main spring of ejector mechanism 40, as will be explained in more detail below. Typically, the injection system is configured so that only one stroke of arming mechanism 25 is required to fully arm ejector mechanism 40. Once pedal 27 and lever 29 are released, lever 29 returns to the position shown in FIG. 1B (e.g., via operation of a return spring). During this tethered mode of operation, injection device 23 remains connected to arming mechanism 25 during administering of injections. This may be desirable in some settings to increase the rate at which injections are administered, by avoiding repeated coupling and decoupling of the injection device and arming mechanism.

Alternatively, as shown in FIG. 1C, injection system 21 may be used in an “untethered” mode. In this mode, injection device 23 is armed by engaging a rearward portion of injection device 23 with arming mechanism 25, so that cable segment 35 is directly engaged with arming mechanism 25 without attachment of intervening cable extension 37. Specifically, as shown in the figure, the rearward portion of injection device 23 is inserted into receiver 43 of arming mechanism 25, which may be shaped, as shown, so as to correspond with rear housing piece 33 and thereby guide the injection device into the proper position when placed in receiver 43. While injection device 23 is thus engaged with arming mechanism 25, operating lever 29 causes cable segment 35 to be pulled, thus arming the device as explained with reference to FIG. 1B. The pedal is then released, and the injection device may be removed from the arming mechanism prior to delivering the injection. In some settings, this untethered mode of operation may be more advantageous than the above-described tethered mode.

FIGS. 2 and 3 depict nozzle assembly 30 as attached to a forward end of ejector mechanism 40. Nozzle assembly 30 and ejector mechanism 40 may be used with any of the embodiments described herein, and may be used in either the untethered mode or tethered mode described above. FIG. 2 shows ejector mechanism 40 in an armed state, while FIG. 3 shows ejector mechanism 40 after it has been discharged to deliver an injection. Ejector mechanism 40 includes an outer housing formed from a cylindrical main body 42, a front shell 44 which may be formed from two halves, a back connector 46 threaded onto a rear portion of main body 42, and a trigger sleeve 48 slidably disposed around a portion of main body 42. As described below, advancing trigger sleeve 48 leftward along an injection axis 50 from the position shown in FIG. 2 releases a locking mechanism in order to discharge the device. Typically, front shell 44 connects to or is formed integrally with front housing piece 34, while trigger sleeve 48 connects to or is formed integrally with rear housing piece 32.

A plunger release guide 52, a firing assembly shoulder 54 and a locking guide 56 are fixedly disposed within the outer housing so that they do not move relative to main body 42 during arming and discharge of the device.

Nozzle assembly 30 may include a nozzle 60 and a skin-tensioning ring 62. Typically, a plunger 64 is slidably disposed within a fluid chamber 66 defined within nozzle 60. Plunger 64 may thus be retracted (i.e., moved to the right in FIGS. 2 and 3) along injection axis 50 to draw a dose of injectable fluid through injection orifice 68 into fluid chamber 66. Forcibly advancing the plunger (i.e. to the left in FIGS. 2 and 3) causes fluid to be expelled from the fluid chamber 66 out through the injection orifice 68, for example to deliver a pressurized needle-free injection of fluid to injection site 24 (FIG. 1A).

Typically, nozzle assembly 30 is configured for selective attachment to and removal from the forward end of ejector mechanism 40. Various structures may be provided to facilitate such attachment and removal, including a nozzle release button 70 (shown in FIGS. 1A and 6 and discussed specifically with reference to FIG. 6), a nozzle slide latch 72, a nozzle release member 74 and a nozzle release spring 76. Operation of these structures, and attachment and removal of nozzle assembly 30, will be described in detail with reference to FIG. 6.

Disposed within ejector mechanism 40 is a firing member or assembly, such as piston assembly 80. As explained in more detail below, piston assembly 80 pulls plunger 64 rearward during arming of the system, and drives the plunger forward during discharge to forcibly eject fluid outward from orifice 68 to deliver the injection. As shown, piston assembly 80 may include a cable piston 82, a choke member 84 and a spring piston 86, all of which are reciprocally movable along injection axis 50 within the interior of ejector mechanism 40. Though these components are formed separately in the depicted example, they may be formed as a single integrated component. A piston spring 90 is disposed between back connector 46 and spring piston 86, so as to urge the piston assembly forward. A cable 100 may be provided to facilitate rearward retraction of piston assembly 80 within ejector mechanism 40. Cable 100 extends between ejector mechanism 40 and arming mechanism 26 (FIG. 1A). One end of the cable terminates in a ball 102 or like anchor, which may be received and held by choke member 84. Referring to FIG. 1A, cable 100 may be slidably disposed within a housing 104 extending between arming mechanism 26 and injection device 22. In alternate embodiments, such as the system of FIGS. 1B and 1C, the injection device may be provided with a short cable segment extending slightly out of the injection device housing. The exposed portion of the cable segment may then be coupled to the arming mechanism, either directly or with an intervening cable extension, as described above.

As piston assembly 80 advances and retracts within ejector mechanism 40, it moves past a locking mechanism 110 configured to selectively maintain piston assembly 80 locked in the armed position shown in FIG. 2. Locking mechanism 110 includes: a slide bushing 112; a slide bushing spring 114 disposed between the slide bushing and firing assembly shoulder 54 and balls 116. Selective locking and releasing of the piston assembly is also facilitated by locking guide 56, and by holes 118 provided in the locking guide to receive balls 116.

Starting from the position shown in FIG. 3, the arming and discharging of the injection device will now be described. First, cable 100 is pulled rearward (i.e., to the right in the figure) by operation of arming mechanism 26 (e.g., by stepping on foot pedal 28 to cause the cable to be pulled). Cable ball 102 is captured by choke member 84, such that pulling of the cable causes piston assembly 80 to retract within ejector mechanism 40 (i.e., move to the right in FIGS. 2 and 3), compressing piston spring 90 until the piston assembly is in the fully retracted and armed position shown in FIG. 2. The forward end of cable piston 82 is operatively coupled with plunger 64, as described in more detail below, such that retraction of the piston assembly causes retraction of plunger 64. Retraction of plunger 64 draws a dose of injectable fluid from an external supply (not shown) into fluid chamber 66 through injection orifice 68. The loading of injectable fluid into fluid chamber 66 from an external supply will be described in more detail below.

Prior to full retraction of piston assembly 80, balls 116 are seated within holes 118 of locking guide 56 and abut an inclined lip portion 120 of slide bushing 112. Any number of balls and corresponding locking guide holes may be employed. For example, three or four balls may be evenly spaced about locking guide 56 (e.g., at 120° or 90° intervals about the circumference of the locking guide). Slide bushing spring 114 is biased to urge slide bushing 112 rearward, i.e., to the right in FIG. 3. However, with balls 116 seated as shown, slide bushing 112 is trapped between slide bushing spring 114 and balls 116 and cannot move.

As piston assembly 80 reaches the fully retracted position shown in FIG. 2, a circumferential locking groove 122 on cable piston 82 comes into alignment with balls 116. At that point, slide bushing spring 114 and inclined lip portion 120 of slide bushing 112 cooperate to push balls 116 radially inward into locking groove 122. Slide bushing 112 is then permitted to slide rearward past balls 116 into the position shown in FIG. 2. In this position of slide bushing 112, balls 116 are prevented from moving radially outward by an inward-facing surface 124 of slide bushing 112. In this position, the interaction between balls 116 and groove 122 prevents the plunger assembly from moving forward, despite the force being exerted due to the compression of piston spring 90.

In the state just described—that is, with piston assembly 80 and plunger 64 retracted and a dose of injectable fluid loaded into fluid chamber 66—the system is armed and ready to deliver an injection. The device may then be discharged by first placing the forward end of nozzle assembly 30 against the injection site (FIG. 1A). The operator then manipulates the device so that rear housing piece 32 is advanced relative to front housing piece 34 to close gap 36. Because rear housing piece 32 is coupled to trigger sleeve 48, advancing the rear housing piece causes the trigger sleeve to push against one or more trigger sleeve pins 130, which extend radially inward into slide bushing 112. Three pins may be provided at equal 120° intervals around the trigger sleeve, or other numbers and arrangements of pins may be employed. Because of the trigger sleeve pins, as rear housing piece 32 and trigger sleeve 48 move forward relative to front shell 44 and front housing piece 34, slide bushing 112 moves forward. As slide bushing 112 moves forward, a space is made available into which balls 116 may move radially outward in response to the sizable forward-directed force being exerted upon piston assembly 80 by piston spring 90.

Once balls 116 have moved radially outward, the balls and locking groove 122 no longer block forward movement of piston assembly 80. Accordingly, piston spring 90 decompresses, causing piston assembly 80 to move forward rapidly and thereby expel fluid from fluid chamber 66 out through injection orifice 68 and into the injection site.

A return spring (not shown), biased against forward movement of trigger sleeve 48 may be provided to return the trigger sleeve to the original pre-injection position. Also, a recess or cavity 140 may be provided within cable piston 82 to prevent the cable from impeding advancement of piston assembly 80 during discharge. Specifically, after piston spring 90 is compressed but prior to delivery of the injection, foot pedal 28 (FIG. 1A) is released after the arming operation. Releasing the foot pedal causes cable 100 to be advanced within housing 104 (FIG. 1A), so that cable ball 102 moves forward away from choke member 84 and into the forward part of cavity 140, as shown in FIG. 3. With cable ball 102 spaced in this manner from choke member 84, piston assembly 80 can advance forward during discharge without being impeded by added drag from cable 100.

Referring now to FIGS. 4 and 5, nozzle assembly 30 will be described in more detail, along with a filling adapter 150 that enables injectable fluid to be loaded into fluid chamber 66 from an external supply. Nozzle assembly 30 and filling adapter 150 may be collectively referred to as nozzle/filling assembly 152. Various components of nozzle/filling assembly 152 are shown exploded apart in FIG. 4 (together with vial adapter 240), with FIG. 5 showing the components of nozzle/filling assembly 152 assembled and ready for use. Nozzle assembly 30 includes a nozzle 60, in which fluid chamber 66 is defined. Lugs 154 are provided at an end of nozzle 60 to facilitate attachment of nozzle assembly 30 to the ejector mechanism, as described below with reference to FIG. 6. Injection orifice 68 is defined at a forward end of nozzle 60.

Typically, nozzle assembly 30 also includes plunger 64, which has an end disposed within fluid chamber 66. As previously described, the plunger is advanced within and retracted from the fluid chamber to draw injectable fluid into, and expel the injectable fluid from, injection orifice 68. Plunger may be provided with an o-ring seal 156, as shown in FIG. 5, or a rubber cap or the like may be provided to cover and seal the entire forward end of the plunger. Additionally, or alternatively, any other appropriate material or structure may be employed to provide a sealing interface between plunger 64 and the interior wall of the nozzle which defines fluid chamber 66.

Nozzle assembly 30 may also include skin-tensioning ring 62 which, as in the present example, is provided as a separate part that is assembled to the rest of the nozzle assembly. Specifically, skin-tensioning ring 62 is slid past lugs 154 and elastically snapped into place so that a portion of skin-tensioning ring 62 is retained in place between snap lip 158 and flange 160 provided on nozzle 60. Skin-tensioning ring 62 typically includes an annular outward-facing surface configured to contact and tension an area (e.g., a patient's skin) surrounding the injection site older

Referring still to FIGS. 4 and 5, filling adapter 150 may include a luer connector 162 and a portion 164 frangibly secured to the forward end of nozzle 60. Luer connector 162 and portion 164 include complementary tooth-like elastic connecting structures 166 and 168 (FIG. 4), enabling luer connector 162 to be snapped into engagement with portion 164. Typically, as in the present example, it will often be desirable that one or more of luer connector 162, portion 164, nozzle 60 and skin-tensioning ring 62 be formed as separate components that are then assembled together. This may be desirable due to manufacturing considerations, such as ease of manufacture and/or the desire or need to manufacture portions of the device from different materials. It will be appreciated however, that these components do not move relative to one another during injections, and thus some or all of them can be molded or otherwise formed integrally as a single piece.

The nozzle/filling assembly of FIG. 5 typically is pre-sterilized and provided to the end-user in the assembled state shown in the figure. Typically, a number of such nozzle/filling assemblies are provided, with each individual assembly being disposable and intended for a single use only, to reduce or limit risk of contamination. On the other hand, ejector mechanism 40 and housing pieces 32 and 34 typically are used for multiple injections, as those parts are often more expensive than nozzle/filling assembly 152, and typically do not come into contact with the injection site or injectable fluid.

Since multiple different nozzle/filling assemblies typically will be used with a single ejector mechanism 40, it will often be desirable to quickly attach nozzle/filling assembly 152 to ejector mechanism 40 to deliver an injection, and quickly remove it after delivery of the injection. FIG. 6 depicts a forward end of ejector mechanism 40 and rearward end of nozzle/filling assembly 152, and illustrates structures involved in coupling and decoupling those components. To couple the components together, the rearward end of nozzle/filling assembly 152 is received within an opening 200 provided in front shell 44. Opening 200 may be provided with a ramp or angled surface 202 to facilitate rotation of nozzle/filling assembly 152 so that nozzle lugs 154 are in a desired rotational orientation relative to ejector mechanism 40.

The end of nozzle/filling assembly 152 and lugs 154 are received within plunger release guide 52 and are pushed against nozzle release member 74 to push the nozzle release rearward into ejector mechanism 40 and thereby compress nozzle release spring 76. A nozzle slide latch 72 is provided within front shell 44 and is urged inward toward injection axis 50 by a spring or springs (not shown) disposed within the front shell. However, prior to insertion of the nozzle assembly, nozzle release member 74 is in a fully forward position, in which it obstructs inward movement of nozzle slide latch 72. Specifically, inward movement of nozzle slide latch 72 is blocked by opposing tabs 204 of nozzle release member 74, which bear against feet 206 of the nozzle slide latch.

Nozzle/filling assembly 152 eventually is pushed far enough into ejector mechanism 40 so that nozzle slide latch 72 is no longer blocked by tabs 204 of nozzle release member 74. Accordingly, nozzle slide latch 72 is urged inward so that a U-shaped opening 208 of the nozzle slide latch embraces the outer diameter of nozzle 60 at a point just forward of lugs 154. When the latch embraces the nozzle in this position, the legs of nozzle slide latch 72 block lugs 154 to prevent removal of nozzle/filling assembly 152 from ejector mechanism 40. Nozzle release button 70 may be provided on an upper portion of front shell 44. Nozzle release button 70 includes two legs 210, and typically is urged outward relative to injection axis 50 by a spring (not shown). Depressing the nozzle release button inward urges release button legs 210 against feet 206 of nozzle slide latch 72 push the nozzle slide latch outward. With nozzle slide latch 72 out of the way of lugs 154, the attached components are ejected by decompression of nozzle release spring 76. From the above, it should be appreciated that nozzle/filling assembly 152 may be engaged and disengaged from ejector mechanism 40 without the operator having to touch the nozzle/filling assembly. This further reduces risk of contamination.

When nozzle/filling assembly 152 is attached to ejector mechanism 40, plunger 64 is also operatively engaged with the ejector mechanism, so that operation of the ejector mechanism causes retraction and advancement of the plunger. In particular, when nozzle/filling assembly 152 is first positioned in the front end of ejector mechanism 40 as described above, cable piston 82 typically is advanced to its forward-most position, as shown in FIG. 3 (e.g., prior to arming of the device and retraction of the cable piston).

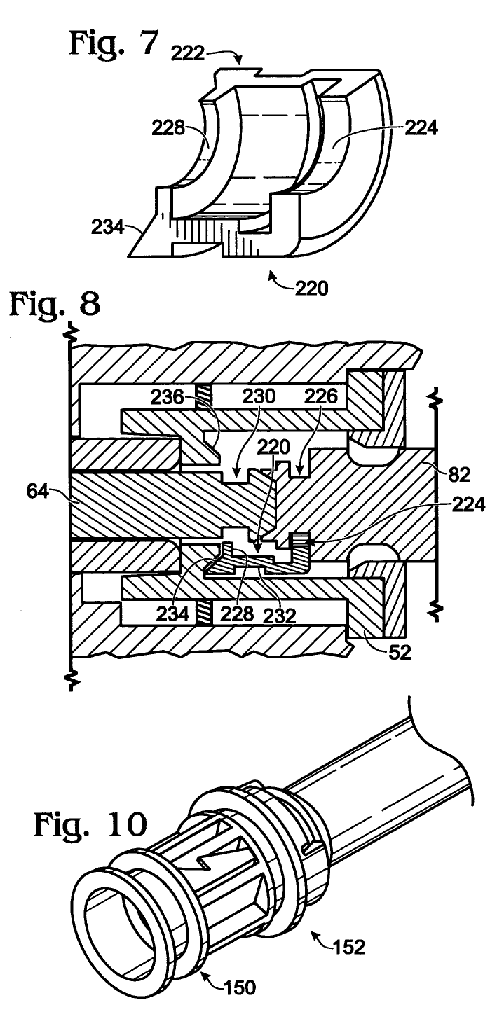

Referring to FIGS. 7 and 8, a plunger coupling device 220 is provided at forward end of cable piston 82. Plunger coupling device 220 may include multiple structures positioned around cable piston 82 and configured to grasp the rearward end of plunger 64. For example, in the depicted embodiment, plunger coupling device 220 includes three collar pieces 222, one of which is shown in FIG. 7. The collar pieces are positioned around the outer diameter of the forward end of cable piston 82. FIGS. 2, 3 and 8 each show one of the collar pieces only for clarity.

As best seen in FIGS. 7 and 8, each collar piece includes an inwardly extending lip 224 which engages a groove 226 at the forward end of cable piston 82 to secure the collar piece to the cable piston. A band, spring or like biasing structure (not shown) is provided to pull the forward ends of collar pieces 222 radially inward, so that a forward lip 228 of each collar piece engages a circumferential groove 230 provided on the rearward end of plunger 64. The biasing structure may take the form of an elastic band positioned within grooves 232 of the collar pieces so as to encircle the collar pieces and pull them radially inward, to thereby grasp the end of plunger 64.

For most of the cable piston's range of motion, collar pieces 222 are urged inward to grasp plunger 64 as just described. However, as shown in FIGS. 7 and 8, when cable piston 82 is at the forward end of its stroke, a ramped portion 234 of collar pieces 222 bears against a ramped portion 236 of plunger release guide 52 to spread the collar pieces outward. Accordingly, despite the countering force provided by the inward biasing of the collar pieces, plunger 64 is released from engagement with cable piston 82 when the cable piston is in the fully advanced position shown in FIG. 8. Thus, the plunger is automatically released during advancement of the piston assembly, and no additional operation is required after the injection to decouple the plunger from the piston assembly.

It will be appreciated that as the cable piston is slightly withdrawn from the position shown in FIG. 8 (e.g., during arming of ejector mechanism 40), collar pieces 222 are permitted to move inward and grasp the end of plunger 64. Indeed, the position shown in FIG. 8 may arise after a fresh nozzle/filling assembly 152 is attached to ejector mechanism 40. Then, as the ejector mechanism is armed, cable piston 82 is retracted and, after a slight bit of retraction, collar pieces 222 grasp the end of plunger 64 so that the plunger continues to move rearward with cable piston 82 to arm the device. The position shown in FIG. 8 also arises after the device has been discharged to deliver an injection. In this case, plunger 64 may be pulled away from the ejector mechanism along with the rest of the nozzle assembly (e.g., by pressing nozzle release button 70 (FIGS. 1 and 6) to eject the spent nozzle assembly 30).

Referring now to FIGS. 1-11, an exemplary method of using the described injection system will be described. First, a fresh, unused nozzle/filling assembly 152 (FIG. 5) is inserted into and secured within the forward end of ejector mechanism 40 (FIGS. 2 and 3), which typically is housed within outer housing pieces 32 and 34 (FIG. 1). The nozzle/filling assembly 152 is secured to ejector mechanism 40 via operation of nozzle slide latch 72 and the accompanying structures described with reference to FIG. 6. Where a large number of injections are to be delivered, a tray or like structure may be loaded with several nozzle/filling assemblies 152 arranged so that the exposed plunger ends are all facing in the same direction. This would allow the operator to grasp the outer housing pieces 34 and 32 and, with a simple one-handed motion, push the housing pieces and ejector mechanism 40 housed therein down onto one of the fresh nozzle/filling assemblies arranged on the tray. This would secure the fresh nozzle/filling assembly 152 in place, as described with reference to FIG. 6.

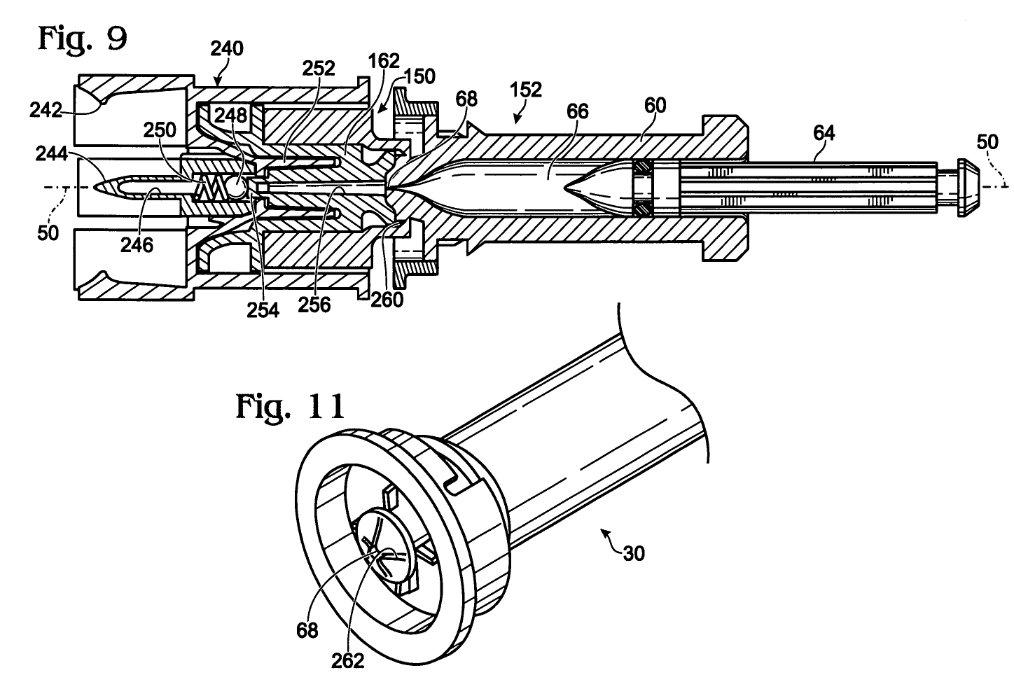

Once nozzle/filling assembly 152 is secured in place, a vial, bottle, container or other external supply of injectable fluid is coupled to the injection system. Typically, the external supply contains multiple doses of injectable fluid, and is used to fill a dose of fluid into each fresh nozzle/filling assembly 152 after it is attached to ejector mechanism 40. For example, FIG. 9 shows vial adapter 240, which is attached to and seals the opening of a vial containing multiple doses of injectable fluid (e.g., a vaccine). Typically, an end of the vial adapter includes structure configured to grip the vial around the vial opening. For example, vial adapter 240 may be snapped onto the vial so that one or more lips 242 grip a rim or neck of the vial. Typically, lip 242 and the arm structures are adapted to tightly grip the vial opening to make it difficult or impossible for the vial adapter to be removed from the vial after it is snapped into place. As shown, the vial adapter includes a piercing member 244, which pierces and extends through the sealed opening of the vial, to allow injectable fluid to pass from the vial into a passage 246 formed in the vial adapter. Passage 246 is sealed via operation of a spring-biased ball valve having a ball 248 and a spring 250. The vial adapter thus seals the vial to maintain injectable fluid within the vial, unless ball 248 and spring 250 are depressed inward (i.e., to the left in FIG. 9).

As shown in FIG. 9, the external supply of injectable fluid is operatively engaged with nozzle/filling assembly 152 by bringing corresponding fittings of vial adapter 240 and filling adapter 150 into engagement. In particular, vial adapter 240 may include a luer connector or fitting 252, which corresponds to the previously-described luer connector 162 of filling adapter 150. Filling adapter 150 includes a protruding portion 254 which pushes ball 248 inward into vial adapter 240, allowing injectable fluid to be drawn into fluid chamber 66 of nozzle 60 upon retraction of plunger 64, as shown in FIG. 9. Specifically, fluid passes from the vial into passage 246, around and past ball 248, into a passage 256 of filling adapter 150, and through injection orifice 68 into fluid chamber 66.

Fluid is drawn into fluid chamber 66 during the previously described arming procedure. Specifically, arming mechanism 26 is operated by stepping on foot pedal 28 (FIG. 1) to pull on cable 100 and thereby retract piston assembly 80 rearward to compress piston spring 90 (FIGS. 2 and 3). Initial rearward motion of the piston assembly causes plunger coupling device 220 (FIGS. 7 and 8) to grasp the rearward end of plunger 64, as described with reference to FIG. 8, so that plunger 64 is retracted to draw injectable fluid from the external supply into fluid chamber 66. When piston assembly 80 and plunger 64 are fully retracted, the device is locked in the armed state via balls 116 and locking groove 122, as described above. Also, after the injectable fluid is loaded into the fluid chamber, the vial and vial adapter 240 are withdrawn from nozzle/filling assembly 152 and engagement with the luer connector of filling adapter 150. Ball 248 then is urged by spring 250 against the valve seat of vial adapter 240 (e.g., the ball moves rightward in FIG. 9), so that the vial adapter seals the external supply of injectable fluid (e.g., the vial of vaccine). Typically, as in the depicted examples, the system is configured so that a single stroke of the arming mechanism (e.g., stepping on the foot pedal once) arms the ejector mechanism and loads fluid into the nozzle assembly.

As previously discussed, nozzle/filling assembly 152 is first pre-assembled and sterilized, and then shipped to the user in the state shown in FIG. 5. Tampering or misuse may be further guarded against by configuring ejector mechanism 40 so that nozzle/filling assembly 152 cannot be attached properly if plunger 64 has been withdrawn. Indeed, the exemplary attachment mechanisms described with reference to FIG. 6 require that the plunger be advanced as shown in FIG. 5 to engage the nozzle/filling assembly with the ejector mechanism. This could potentially discourage unwanted or improper attempts to pre-fill the device, for example prior to attaching the nozzle/filling assembly to the ejector mechanism.

The injection system may be configured so that, once the device is armed with injectable fluid loaded into fluid chamber 64, filling adapter 150 must be broken off the end of nozzle assembly 30 to successfully inject the fluid that has been loaded into fluid chamber 64. FIG. 10 shows nozzle/filling assembly 152 after the vial and vial adapter 240 have been withdrawn, but before filling adapter 150 has been broken away from nozzle assembly 30. FIG. 11 shows the nozzle/filling assembly after the filling adapter has been broken away, such that only nozzle assembly 30 remains. FIGS. 2 and 3 also show the system after filling adapter 150 has been broken off.

Breakage may be facilitated by a frangible connection 260 between filling adapter 150 and nozzle assembly 30, as shown in FIGS. 5 and 9. The frangible connection typically is implemented by thinning or otherwise weakening material at the desired point of breakage, or through other methods/structures that produce breakage in a desired location when sufficient force is applied. In the depicted example, the desired point of breakage occurs lengthwise at or very near the point at which the skin-tensioning ring contacts the injection site. Accordingly, everything forward of the injection orifice and skin-tensioning ring is broken away. Nonetheless, as shown in FIGS. 2 and 3, it may be desirable for the tip of the injection device around the injection orifice to be slightly forward of skin-tensioning ring 62. After the filling adapter is removed, the injection orifice and skin-tensioning ring may be placed into contact with the surface of the injection site just prior to triggering of the injection. The frangible connection typically is designed to break upon application of a torsional force of predetermined magnitude, while at the same time being designed to withstand anticipated axial forces (e.g., along injection axis 50), such as might be expected to occur during assembly, storage, filling, etc.

After filling adapter 150 is broken off, the loaded and armed device is positioned over an injection site, as shown in FIG. 1. The injection system is then pressed onto the injection site to trigger the injection. Outer housing pieces 32 and 34 may also be squeezed together to trigger the injection, as described above, in order to reduce pressure on the surface of the injection site. Various safety or disabling mechanisms may be provided to prevent triggering of an injection unless certain conditions are satisfied. For example, an obstructing member may be interposed in the gap between trigger sleeve 48 and front shell 44 (FIGS. 2 and 3). Such an obstructing member would have to be withdrawn from the gap in order to permit the relative motion between the trigger sleeve and front shell that triggers the injection. Withdrawal of the obstructing member could be prevented unless an additional user operation was performed, and/or unless various safety and/or sterilization conditions were satisfied, the satisfaction of which could be determined through sensors or other methods.

Once the injection has been delivered, the spent nozzle assembly 30 is ejected via operation of nozzle release button 70, and a new unused nozzle/filling assembly 152 may be filled and used to deliver another injection using the process described above. As previously discussed, the used nozzle assembly typically is discarded just by pressing the nozzle release button, and is not touched or otherwise manipulated by the operator.

As discussed above, the injection system of the present description typically is configured so that, to provide an injection, the operator must first perform an act which renders the nozzle assembly incapable of being reused. More specifically, the injection cannot be performed in the described exemplary system unless the user breaks off filling adapter 150. The nozzle/filling assembly is configured so that the filling adapter cannot be reattached after it is removed (e.g., because the adapter's connection to the nozzle assembly is structurally broken). Once the filling adapter is broken off, there is no way to refill nozzle assembly 30 from the external supply of injectable fluid, because the nozzle assembly itself (e.g., without filling adapter 150) has no fitting or other structure to operatively engage the fitting on the vial (e.g., luer fitting 252 of vial adapter 240). Accordingly, in the described example, the spent nozzle assembly is useless and must be discarded. Because the operator is effectively prevented from reusing the nozzle assembly, which typically is intended to be a single-use disposable item, the risk of contamination may be further reduced.

Furthermore, as shown in FIG. 11, nozzle 30 may be provided with seal-defeating structures adjacent injection orifice 68 to further guard against refilling attempts. In the depicted example, the seal-defeating structures are implemented as channels 262 formed in the nozzle and extending radially outward from injection orifice 68. Channels 262 are adapted to compromise attempts to establish a seal against the outer surface of nozzle 30 in the area around injection orifice 68, making it difficult to force or draw fluid into the fluid chamber through the injection orifice after the filling adapter has been removed.

Also, referring to FIGS. 4, 5, 9 and 12, vial adapter 240 and filling adapter 150 typically are adapted so that the fluid pathways are disposed in interior locations, with various outer structures protecting the pathways from contact with the system operator or other potential sources of contamination. For example, connecting structures 166 and 168 (FIG. 4) surround and protect the fluid pathway of filling adapter 150, which is aligned along the injection axis and includes the interior of luer fitting 162 and passage 256. Vial adapter 240 similarly includes a protected fluid pathway (e.g., passage 246 and the interior of luer fitting 252), which is aligned with the injection axis in the center of the vial adapter.

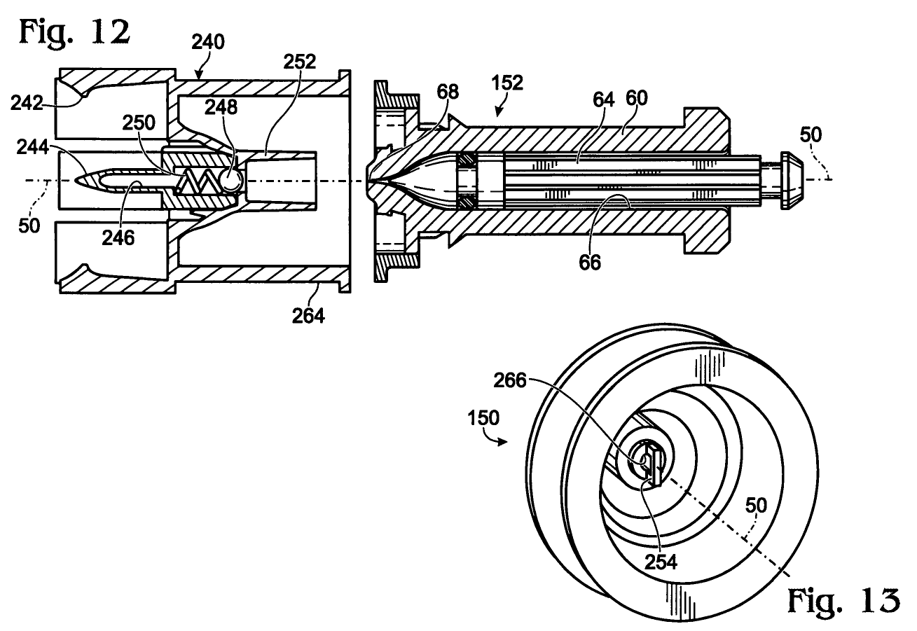

As shown in FIG. 12, the fluid pathway and luer fitting 252 are recessed and disposed within an outer protective shroud 264. Shroud 264 provides further protection against contamination and/or misuse, by preventing the fluid pathway of vial adapter 240 from coming into contact with a spent nozzle assembly or with work surfaces or other sources of contamination. This feature is shown in FIG. 12, which illustrates an improper attempt to couple the external supply of injectable fluid with nozzle/filling assembly 152 after an injection has been delivered. At this point, because the filling adapter has been broken away to deliver the injection, no structure remains on the nozzle that can be engaged with the vial adapter luer fitting (e.g., fitting 252). In addition, shroud 264 prevents the vial adapter fluid pathway from being brought into contact with the injection orifice.

As previously discussed, nozzle/filling assembly 152 typically is configured to prevent and/or interfere with delivery of an injection prior to detachment of filling adapter 150. The prevention or interference may be accomplished by blocking the injection and/or preventing the injection orifice from being brought into sufficiently close contact with the surface of the injection site. Referring first to FIG. 9, protruding portion 254 of filling adapter 150 provides an obstruction which is aligned along injection axis 50. Because the injection axis extends through the obstruction, attempts to deliver an injection while the filling adapter is in place will be unsuccessful. Specifically, the obstruction will block the injection and diffuse the expelled fluid prior to entry into the injection site, so that the stream of fluid is unfocused and insufficiently pressurized to penetrate the surface of the injection site.

Although the obstruction is positioned within the injection axis, filling adapter 150 nonetheless permits fluid to pass around the obstruction and into fluid chamber 66 via injection orifice 68 during filling. Specifically, during filling, the fluid is drawn from the external supply and passes around ball 248. The fluid then deviates slightly off of injection axis 50 and around obstruction 254 into passage 256. Specifically, referring to FIGS. 9 and 13, obstruction 254 is positioned centrally over the opening of passage 256. The obstruction is aligned to block the injection axis, but does not completely cover the opening of passage 256. Accordingly, holes 266 adjacent obstruction 254 allow fluid to pass around the obstruction during the filling operation described above. Thus, even though obstruction 254 prevents injection attempts prior to removal of filling adapter 150, the obstruction does not interfere with filling.

In addition to obstructing attempted injections, the filling adapter also may be positioned relative to the injection orifice so as to make an injection impossible without detaching the filling adapter. Specifically, the depicted filling adapter makes it impossible to bring the injection orifice adjacent to or in close contact with the surface of the injection site. Accordingly, due to the distance between the injection orifice and injection site, the expelled fluid would be dispersed and unfocused, and without sufficient pressure to penetrate the injection site. Typically, this would occur even without the above-described interference of obstruction 254. However, once the filling adapter is removed, the injection axis is no longer obstructed and the injection orifice may be placed onto the injection site to deliver the injection.

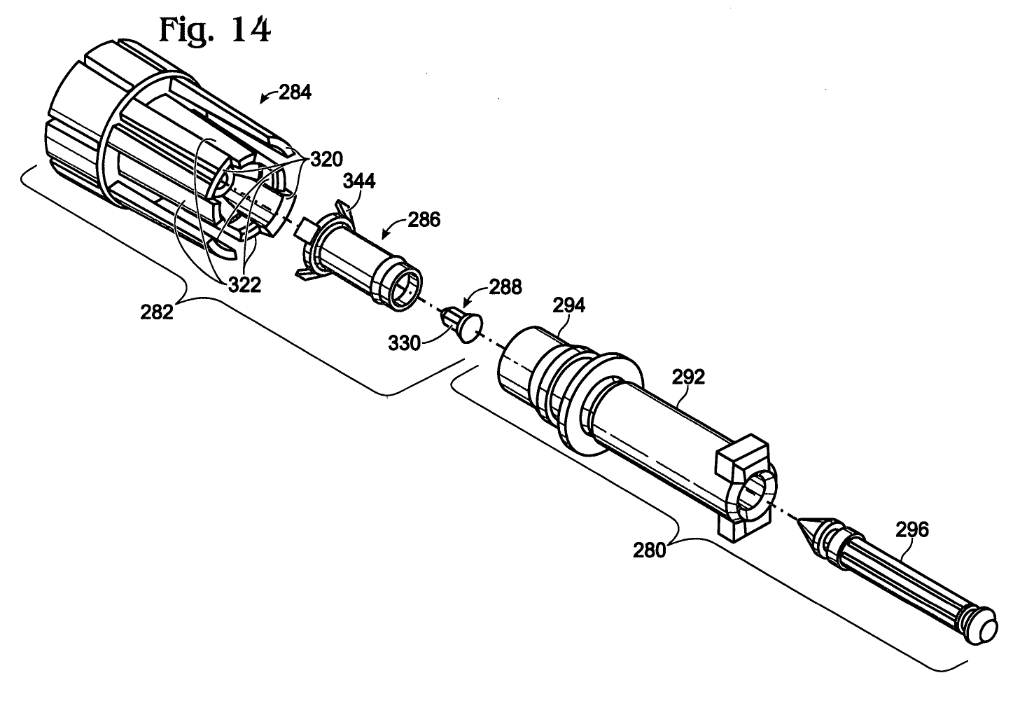

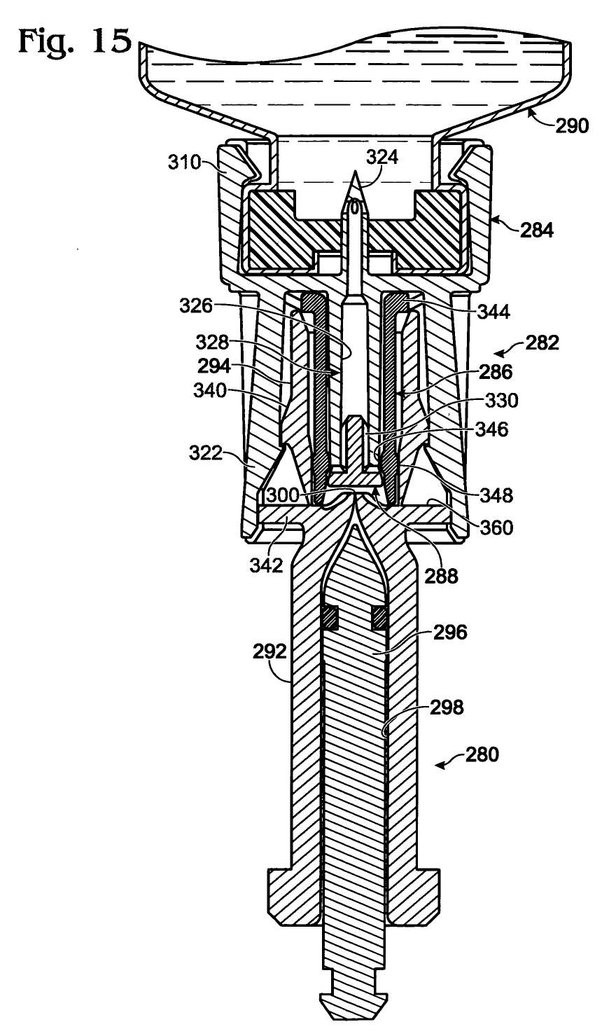

FIGS. 14 and 15 depict further embodiments of a nozzle/filling assembly 280 and vial adapter 282 according to the present description. Vial adapter 282 may include a main body 284, inner valve sleeve 286 and plug 288. As in the previously-described examples, vial adapter 282 typically is attached to and carried on a multiple-dose container (e.g., vial 290) of injectable fluid. Nozzle/filling assembly 280 may include a nozzle 292, a filling adapter 294 secured to the front end of the nozzle, and a piston 296 slidably disposed within a fluid chamber 298 of the nozzle. Nozzle/filling assembly 280 may be engaged with, and disengaged from, an injector device such as ejector mechanism 40, as previously described with reference to nozzle/filling assembly 152.

As in the previous examples, nozzle/filling assembly 280 typically is provided to the end user in a ready-to-fill state. In this state, the nozzle/filling assembly may be operatively engaged with vial adapter 282 to perform the filling operation, in which a dose of injectable fluid is drawn from vial 290 through injection orifice 300 and into fluid chamber 298 of nozzle 292. To allow the injection to go forward, filling adapter 294 is broken away from nozzle 292. Similar to the above-described embodiments, filling adapter 294 is specially configured to operatively engage with vial adapter 282 to perform the filling operation. Typically, the system is configured so that filling cannot occur after filling adapter 294 is broken away. Thus, as before, a single simple step permits the injection to go forward, while simultaneously disabling the ability to refill nozzle 292.

Main body 284 of vial adapter 282 includes a vial gripping section 310 adapted to grip a vial of injectable fluid (e.g., vial 290), and several fingers extending axially away from the gripping section. The extending structures may include relatively rigid fingers 320 and relatively flexible fingers 322. In the depicted embodiment, there are four rigid fingers, with a flexible finger disposed between each rigid finger, for a total of eight fingers, though it should be appreciated that different numbers of fingers may be employed in various configurations.

Vial adapter 282 includes a piercing member or spike 324 configured to pierce a sealed opening of vial 290. Openings are provided on piercing member 324 to enable injectable liquid from vial 290 to flow into a central channel 326 defined within a cylindrical member 328 extending away from gripping section 310 between fingers 320 and 322. Plug 288 is fitted snugly into the distal end of cylindrical member 328. As indicated in FIGS. 14 and 15, plug 288 includes channels 330 configured to permit fluid to be drawn out of central channel 326 and into the area around injection orifice 300 of nozzle 292. As will be explained in more detail, inner valve sleeve 286 may be axially movable between a position, in which it seals off channels 330, and an unsealed position, in which liquid is permitted to pass out through the channels to injection orifice 300.

Referring specifically to FIG. 15, to fill the device, nozzle/filling assembly 280 is first inserted into and received within vial adapter 282. Prior to this, nozzle/filling assembly 280 may first be secured within an injector device or mechanism, such as ejector mechanism 40, using the previously-described structures and methods. As nozzle/filling assembly 280 is inserted into vial adapter 282, a ramped portion 340 on the outer diameter of filling adapter 294 bears against flexible fingers 322, urging them outward. Flexible fingers 322 are urged far enough outward by filling adapter 294 so that the flexible fingers are pushed beyond the outer edges of a flanged portion 342 of nozzle 292, thereby allowing the nozzle/filling assembly to be inserted further into vial adapter 282.

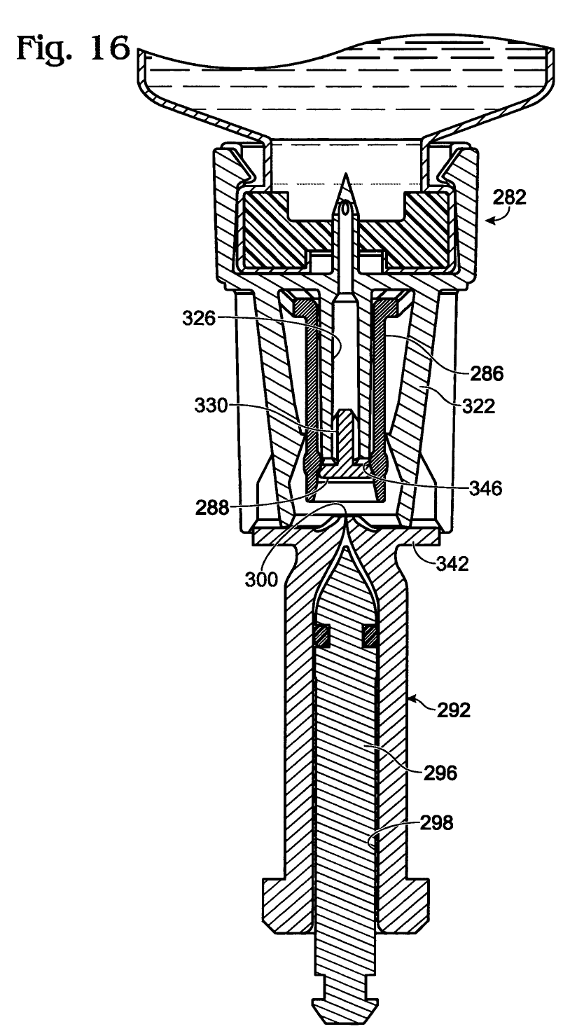

Inserting nozzle/filling assembly 280 into vial adapter 282 also causes a forward end of nozzle 292 to push against the distal end of inner valve sleeve 286. Prior to contact with nozzle 292, inner valve sleeve 286 is biased axially away the vial-gripping portion of vial adapter 282 by resilient feet 344 provided on the proximal end of inner valve sleeve 286. In this initial position (shown in FIG. 16), an annular protruded area 346 on the inner diameter of inner valve sleeve 286 seals channels 330 formed in plug 288, thereby preventing liquid from passing out of central channel 326.

The insertion of nozzle/filling assembly 280 into vial adapter 282 pushes the inner valve sleeve 286 axially toward vial 290, compressing feet 344 and moving the sleeve so that the annular protruded area 346 does not seal channels 330 (FIG. 15). Piston 296 may then be drawn back to draw a dose of injectable liquid into fluid chamber 298 of nozzle 292. To create suction, the outer diameter of inner valve sleeve 286 may also be provided with an annular protruded area 348 to seal against the inner diameter of filling adapter 294.

After piston 296 has been withdrawn to draw in a dose of injectable fluid, filling adapter 294 may be broken away from nozzle 292. Typically, nozzle/filling assembly 280 is manufactured so that there is a frangible connection 360 between filling adapter 294 and nozzle 292 at the desired breaking point. Typically, after the filling adapter is broken away, it cannot be reattached by the user to the nozzle.

Referring now to FIG. 16, it will be appreciated that the described exemplary system prevents filling after the filling adapter has been broken away. Specifically, the figure depicts a non-compliant attempt to engage vial adapter 282 with nozzle 292 after the filling adapter has been broken away from the front of nozzle 292 (e.g., after an injection has been delivered). As shown, flexible fingers 322 of vial adapter 282 are biased inward so as to block the flanged portion 342 of nozzle 292 surrounding injection orifice 300. Since filling adapter 294 (FIGS. 14 and 15) has been broken away, no structure remains to spread the flexible adapter structure outward away from the blocking position to allow further axial movement of nozzle 292 toward vial adapter 282.

Because the flexible fingers act as a blocking mechanism or outer protective shroud that maintains nozzle 292 spaced apart from the end of inner valve sleeve 286, the respective fluid paths of vial adapter 282 and nozzle 292 are prevented from coming into contact, thereby guarding against contamination. Also, the nozzle is prevented from pushing against the end of inner valve sleeve 286, such that the nozzle cannot push the inner valve sleeve inward to disable the sealing of channels 330 by annular protruded area 346. Furthermore, because filling adapter 294 has been removed, a seal cannot be established to seal an enclosed area between the fluid paths. Accordingly, it should be appreciated that the removal of filling adapter 294 guards against contamination, prevents refilling and otherwise protects against unintended use.

As in the previous examples, the device depicted in FIGS. 14-16 is configured to prevent delivery of an injection until the filling adapter is broken away and the refilling capability disabled. Specifically, the filling adapter may be disposed on the nozzle and sized so that the injection orifice is sufficiently spaced from the injection site so as to prevent an effective injection from occurring.

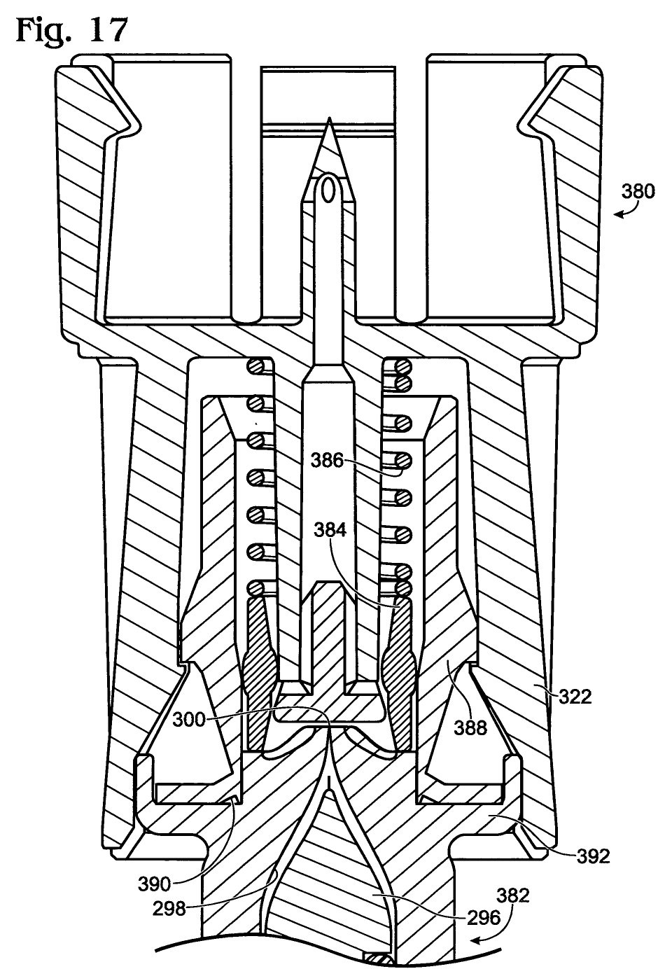

FIG. 17 depicts further alternate embodiments of a vial adapter 380 and nozzle/filling assembly 382 according to the present disclosure. Vial adapter 380 differs from the vial adapter of FIGS. 14-16 in that it includes an alternate inner valve sleeve 384 which is biased into a sealed position by a spring 386. In the sealed position (not shown), the inner diameter of valve sleeve 384 seals channels 330 of plug 288. As in the example of FIGS. 14-16, nozzle/filling assembly 382 includes a filling adapter 388 that spreads flexible fingers 322 apart to enable the components to be positioned axially close enough to one another to defeat the sealing of channels 330 and create suction to allow fluid to be drawn into fluid chamber 298 upon retraction of piston 296. During retraction of piston 296, the outer diameter of valve sleeve 384 seals against the inner diameter of filling adapter 388 to create suction.

Also, nozzle/filling assembly 382 differs from that of FIGS. 14-16 in that frangible connection 390 is in a recessed location relative to injection orifice 300. Specifically, the frangible connection is spaced axially away in a rearward direction (e.g., rearward along the injection axis) from the generally planar area at the forward end of nozzle 392 that is placed onto the injection site during delivery of an injection. This may be desirable in certain applications, to ensure that sharp edges or other irregularities resulting from breakage are prevented from coming into contact with the injection site (e.g., a patient's skin). Also, as indicated, filling adapter 388 may be fabricated as a separate piece, rather than integrally formed with nozzle 392. In the depicted example, the separate filling adapter piece may be ultrasonically welded to nozzle 392 or secured in place with any other desired method.

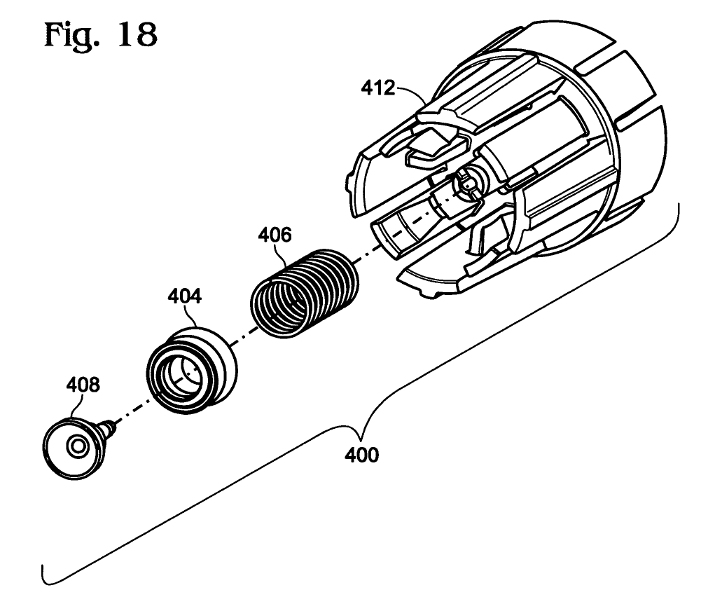

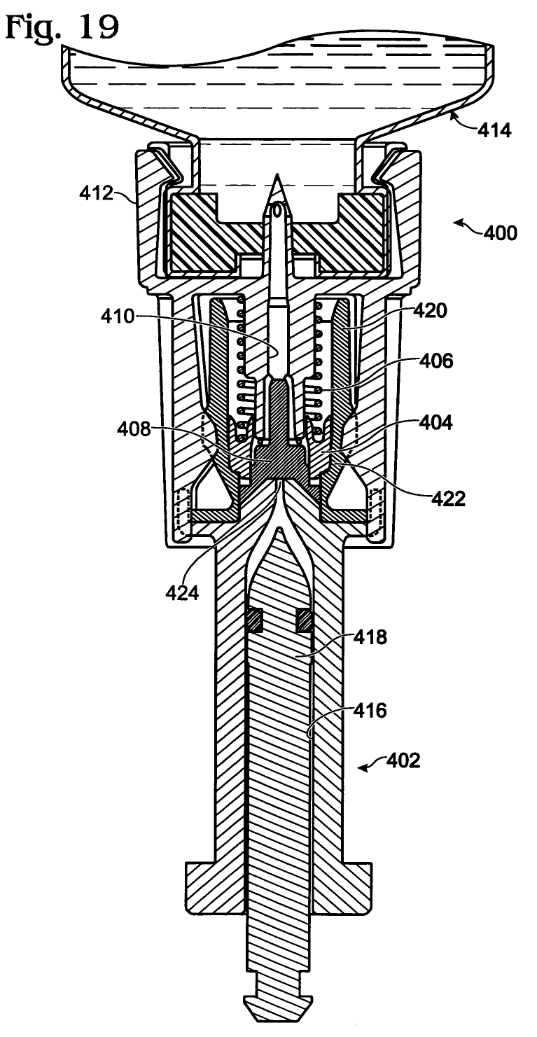

FIGS. 18 and 19 depict further alternate embodiments of a vial adapter 400 and nozzle/filling assembly 402 according to the present disclosure. Similar to the example of FIG. 17, vial adapter includes a valve sleeve 404 which is biased (downward in FIG. 19) into a sealed position by a spring 406. A plug 408 is fitted into a cylindrical passage 410 of the vial adapter main body 412. As in the previous exemplary embodiments, plug 408 includes channels through which fluid can flow from vial 414 out through passage 410 and out of the vial adapter (e.g., into variable volume fluid chamber 416 in which plunger 418 is disposed), however a lower end of sleeve 404 seals these channels until the sleeve is moved out of the sealing position (e.g., moved upward against the spring tension via engagement of the vial adapter with an appropriately shaped filling adapter).

FIG. 19 depicts engagement of nozzle/filling assembly 402 with vial adapter 400. As shown, filling adapter 420 may include on its inner diameter a circumferential ledge 422 sized to bear against a lower portion of valve sleeve 404 when the components are brought together. This urges valve sleeve 404 upward against the force of spring 406, as shown in the figure, such that fluid is now permitted to pass from passage 410, through the channels in plug 408, and into the injection device through injection orifice 424.

It will be appreciated that the nozzle/filling assembly 402 and vial adapter 400 provide similar advantages to the other embodiments discussed herein. In particular, filling adapter is configured so that it is frangibly connected to the nozzle, and must be broken away before an injection can be administered. As in the other embodiments, this breaking of the filling adapter prevents reuse by disabling the ability to refill the device. Specifically, once the filling adapter has been removed, the nozzle is no longer shaped to engage the opening of the vial adapter and actuate the adapter valve seal. Also, the vial adapter has an outer structure, as in previous embodiments, that acts as a protective shroud to protect the fluid pathway and reduce risk of contamination.

FIG. 20 depicts in more detail the alternate injection device 23 shown in FIGS. 1B and 1C. Injection device 23 is similar in many respects to the injection device of FIG. 1A. In particular, injection device 23 includes an outer housing that may be constructed of multiple pieces which are moveable relative to one another in order to actuate injections. For example, back housing 33 is attached to, or formed integrally with, trigger sleeve 48 of ejector mechanism 40, which is disposed within the injection device housing, as in the prior embodiments. Front ejector sleeve or housing 45 of the ejector mechanism is connected to, or formed integrally with, front outer housing 31. Thus, similar to the previous embodiments, after ejector mechanism 40 is armed, discharge may be affected by moving housing pieces 31 and 33 relative to one another.

In contrast to the prior embodiment, injection device 23 may be provided with a short cable segment 35, as previously described. Cable segment 35 enables the injection system to be used in either of two different operating modes. In the tethered operating mode (FIG. 1B), a cable extension 37 is secured to anchor 35a of cable segment 35. The other end of cable extension 37 to arming mechanism 25, such that operation of lever 29 causes cable segment 35 to be pulled, thus compressing spring 90 and arming the ejector mechanism.

Referring first to FIG. 1B and to the end of cable extension 37 nearest to the injection device 23, cable housing 39 is attached to a fitting 41 or other structure adapted to receive or otherwise accommodate the rearward end of the injection device (e.g., the rearward end of back housing piece 33). Extension cable 37 and the cable segment extending out of injection device 23 (i.e., cable segment 35) are secured together via a link 49 or other appropriate device. The end of housing 39 is thus secured to or against the rearward end of back housing 33, while cable extension 39 and cable segment 35 are permitted to slide within housing 39 and fitting 41 upon application of pulling force to the cables.

The opposing end of cable housing 39 may be secured to arming mechanism 25, as shown in FIG. 1B. Typically, the end of extension cable 39 will extend at least partially through receiver 43 into the interior of arming mechanism 25, so that the arming mechanism can grasp or otherwise engage an end of cable extension 39. Arming mechanism 25 typically is configured so that depression of lever 29 causes the arming mechanism to grasp and pull downward a ball or like anchor provided on the end of cable extension 39. This in turn causes cable segment 35 to be pulled so as to compress spring 90 and thereby arm ejector mechanism 40.

Referring more particularly to FIGS. 21, 22 and 23, it will be appreciated that arming mechanism 25 may include a mechanism or structure configured to selectively engage and release the portion of the cable inserted into the arming mechanism. This selective attachment allows the inserted cable portion to be quickly and easily disconnected from the arming mechanism, which may be particularly advantageous when using the system in the untethered mode.

In the depicted exemplary embodiment, arming mechanism 25 includes a latch 430 pivotally attached or linked to a reciprocating member 432. Reciprocating member 432 is disposed within a vertical housing portion 434 of arming mechanism 25, and is operatively coupled with lever 29 so that depressing the lever causes the reciprocating member to move downward relative to housing portion 434. Conversely, upward motion of the lever causes reciprocating member 432 to move upward within housing portion 434. Typically, a spring or other mechanism is provided so that after lever 29 is depressed, it automatically returns upward to its original position shown in FIG. 21 upon being released.

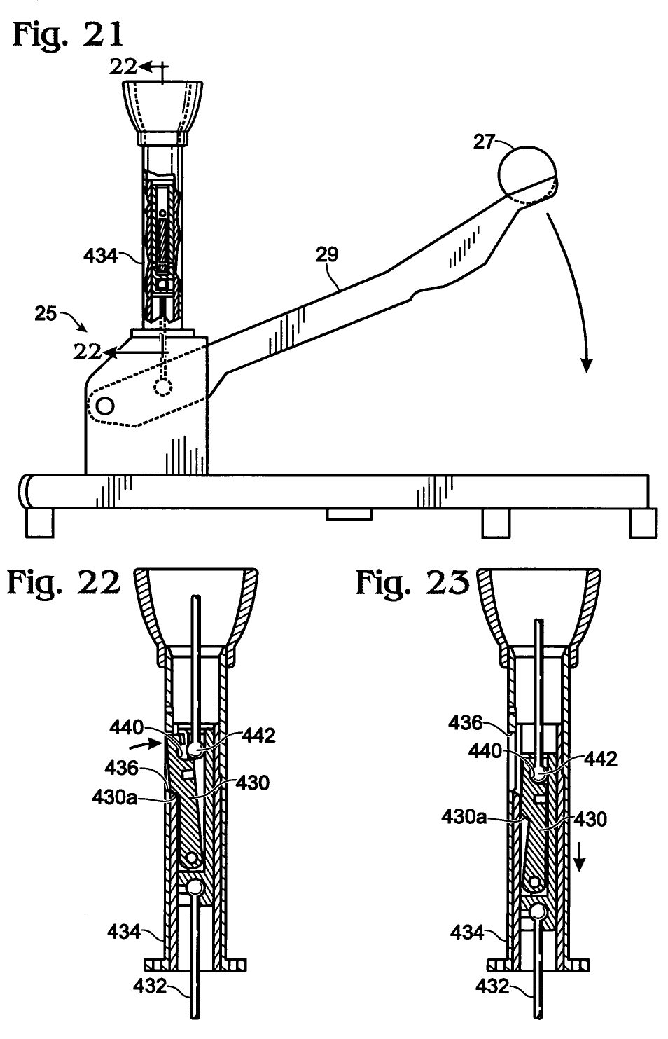

Because latch 430 is linked to reciprocating member 432, it also moves upward and downward in response to movement of lever 29 and pedal 27. In addition to this reciprocating action, the pivotal connection between latch 430 and reciprocating member 432 enables the latch to pivot back and forth as it moves upward and downward. Specifically, a spring or like mechanism biases an upper end of the latch outward, such that when the reciprocating member is in its uppermost position (shown in FIG. 22 and corresponding to the upper, un-depressed position of lever 29), the upper end of latch extends outward into a recess or opening 436 formed in or through wall of vertical housing portion 434. A ramped portion or like feature 430a on latch 430 is configured to interact with vertical housing portion 434, and more particularly with an edge of opening 436, so that, as the reciprocating member and latch move downward in response to depression of lever 29, the latch is forced to pivot inward.

As shown in FIGS. 22 and 23, the upper end of latch includes a socket 440 for receiving cable anchor 442. When latch 430 pivots inward as just described (FIG. 23), socket 440 surrounds cable anchor 442 such that the end of the cable is grasped by the latch. Continued downward movement of reciprocating member and latch 430, as occurs when lever 29 is depressed, thus causes cable extension 37 and cable segment 35 to be pulled, to compress spring 90 and thereby arm the injection device. In the tethered mode of operation, arming and discharge of the device is carried out without decoupling the injection device from the arming mechanism.

The pivoting action of link 430 is particularly advantageous when the injection device is decoupled from the arming mechanism to deliver injections (e.g., untethered mode). In untethered mode, injection device 23 may be inserted so that a rearward portion of back housing piece 33 is positioned within receiver 43, which may be secured to the upper end of vertical housing portion 434 of arming mechanism 25. When the injection device is thus placed into arming mechanism 25, cable segment 35 extends into arming mechanism 25, so that cable anchor 35a (FIG. 20) may be selectively grasped by latch 430 when lever 29 is depressed. Thus, the injection device is armed in the same manner as the previous example, but without the intervening cable extension 37.

After arming, the injection device may be removed and then used to administer the injection. Repeated use of the arming mechanism is thus very convenient, and does not require special tools or actions beyond placement of the injection device and operation of lever 29. Because latch 430 pivots automatically with movement of lever 29, no additional steps are needed to secure cable segment 35 to arming mechanism 25. Once the injection device is armed and pedal 27 is released, latch 430 automatically pivots out of the way, allowing injection device 23 to be freely removed from engagement with the arming mechanism 25.

From the above, it will be appreciated that the exemplary injection systems described herein may provide numerous advantages, particularly in mass immunization settings or other applications where multiple recipients are to be provided with injections. The system is easy to operate and can be used to quickly deliver injections, while minimizing contamination risks. In particular, the nozzle/filling assembly is easily attached with a single motion by causing it to be inserted into the ejector mechanism, which automatically locks the nozzle/filling assembly and ejector mechanism into engagement.

The filling adapter may be integrated with or pre-assembled with the nozzle, and thus no extra steps are required to prepare the device for the filling operation. A vial of injectable fluid is simply engaged with an end of the injection device (e.g., by engaging the corresponding luer fittings), and the device is then armed and filled with a single step by operating arming mechanism 26. The nozzle does not need to be pre-filled, and a filling station or other complex on-site system for filling nozzles is not required. Once the dose is loaded and the injection device is armed, the filling adapter may be quickly and easily broken off, and the injection may be delivered to the recipient. As described above, the need to break off the filling adapter prior to delivering the injection limits risk of reuse or refilling of the nozzle assembly, to thereby reduce the possibility of cross contamination and of contamination of the external supply used to fill the device. Also, all of the disposable fluid-contacting components may be quickly and easily discarded at the end of the injection (e.g., by operating nozzle release button 70).

While various embodiments and arrangements of a needle-free injection system and method have been shown and described above, it will be appreciated that numerous other embodiments, arrangements, and modifications are possible and are within the scope of the invention. The foregoing description should be understood to include all novel and non-obvious combinations of elements described herein, and claims may be presented in this or a later application to any novel and non-obvious combination of these elements. The foregoing embodiments are illustrative, and no single feature or element is essential to all possible combinations that may be claimed in this or a later application.

1. A needle-free injection system, comprising:
a fluid chamber configured to contain a quantity of injectable fluid;
a nozzle including an injection orifice fluidly coupled with the fluid chamber;
an ejector mechanism including a member moveable between a disarmed position and an armed position, where the member is biased toward the disarmed position such that, upon release of the member from the armed position, the member is driven toward the disarmed position, thereby creating a force which causes injectable fluid to be forcibly ejected from the fluid chamber out of the injection orifice; and
an arming mechanism include a lever configured to be operatively coupled with the member and selectively operable to arm the ejector mechanism by causing the member to be moved into the armed position, where the arming mechanism is configured to fully effect such arming with a one-stroke arming motion, in which the lever is moved once from a first position to a second position.

2. The system of claim 1, where the ejector mechanism includes a spring configured to be compressed when the member is moved into the armed position, the spring being further configured to decompress to drive the member toward the disarmed position.

3. The system of claim 2, further comprising a cable segment secured to the member, the arming mechanism being configured so that operation of the lever causes the cable segment to be pulled to move the member into the armed position and thereby compress the spring.

4. The system of claim 3, where the arming mechanism is configured to pull the cable segment upon operation of the lever and, upon release of the lever, automatically release the cable segment so as to permit removal of the cable segment from engagement with the arming mechanism.

5. The system of claim 4, where the arming mechanism includes a pivoting latch configured to automatically grasp and release an anchored end of the cable segment.

6. The system of claim 1, where the arming mechanism is configured to permit repeated arming and discharge in either a tethered mode, in which the ejector mechanism remains operatively coupled with the arming mechanism, or an untethered mode, in which the ejector mechanism is decoupled from the arming mechanism prior to each discharge.

7. A needle-free injection system, comprising:
a fluid chamber configured to contain a quantity of injectable fluid;
a nozzle including an injection orifice fluidly coupled with the fluid chamber;
an ejector mechanism including a member moveable between a disarmed position and an armed position, where the member is biased toward the disarmed position such that, upon release of the member from the armed position, the member is driven toward the disarmed position, thereby creating a force which causes injectable fluid to be forcibly ejected from the fluid chamber out of the injection orifice; and
an arming mechanism selectively operable to cause the member to be moved into the armed position, the ejector mechanism and arming mechanism being configured so as allow an operator of the injection device to decouple the ejector mechanism from the arming mechanism after arming of the ejector mechanism and prior to administering an injection.

8. A needle-free injection system, comprising:
a fluid chamber configured to contain a quantity of injectable fluid;
a nozzle including an injection orifice fluidly coupled with the fluid chamber;
an ejector mechanism including a member moveable between a disarmed position and an armed position, where the member is biased toward the disarmed position such that, upon release of the member from the armed position, the member is driven toward the disarmed position, thereby creating a force which causes injectable fluid to be forcibly ejected from the fluid chamber out of the injection orifice; and
an arming mechanism selectively operable to cause the member to be moved into the armed position, where the ejector mechanism and arming mechanism are configured for use in either a tethered mode or an untethered mode,
where in the untethered mode, the ejector mechanism is decoupled from the arming mechanism after arming of the ejector mechanism and prior to administering an injection, and
where in the tethered mode, the ejector mechanism and arming mechanism remain operatively coupled together during administering of an injection.

9. The system of claim 8, further comprising a spring configured to be compressed upon arming of the ejector mechanism, and where the system is configured so that decompression of the spring causes injectable fluid to be forcibly ejected out the injection orifice.

10. The system of claim 9, where the ejector mechanism further includes a piston member coupled to a cable, where pulling the cable causes the piston member to retract and thereby compress the spring in order to arm the ejector mechanism.

11. The system of claim 10, where the cable is operatively coupled to the arming mechanism via a cable anchor disposed within the arming mechanism, and where the arming mechanism is configured to automatically grasp the cable anchor during operation of an actuator of the arming mechanism, and then automatically release the cable anchor upon release of the actuator, to freely permit removal of the cable anchor from the arming mechanism after arming has occurred.

12. The system of claim 8, further comprising a filling adapter secured to the nozzle adjacent the injection orifice and configured to enable an external supply of injectable fluid to be fluidly coupled with the injection orifice.

13. The system of claim 12, where the filling adapter is frangibly attached to the nozzle such that the filling adapter cannot be reattached to the nozzle after being broken away from the nozzle, and where the needle free-injection system is configured to prevent delivery of an injection from the injection orifice into an injection site until the filling adapter is broken away from the nozzle.

14. The system of claim 12, where the needle-free injection system is configured to prevent delivery of an injection into an injection site until the ability of the filling adapter to enable filling of the injection device has been disabled.

15. A needle-free injection system, comprising:
a spring-powered injection device, including a fluid chamber for containing injectable fluid and an injection orifice fluidly coupled with the fluid chamber, the injection device further including a spring configured to be compressed during arming of the injection device, the spring-powered injection device being configured to forcibly eject fluid from the fluid chamber out through the injection orifice during decompression of the spring; and
an arming mechanism configured to be operatively engaged with the injection device so that the spring of the injection device is mechanically coupled with the arming mechanism, the coupling of the injection device and arming mechanism being such that operation of an actuator of the arming mechanism causes the spring of the injection device to be compressed.

16. The system of claim 15, where the system is further configured to permit discharge of the injection device while the injection device remains operatively engaged with the arming mechanism, or after the injection device has been disengaged from the arming mechanism.

17. The system of claim 16, where the injection device includes a reciprocating member configured to act against the spring and retract during arming of the injection device, the reciprocating member including a connector configured to enable the arming mechanism to be operatively coupled with the reciprocating member and cause the reciprocating member to be forcibly retracted upon operation of the actuator of the arming mechanism.

18. The system of claim 17, where the connector includes a cable segment with an anchored end that extends out of a housing of the injection device.

19. The system of claim 18, further comprising a cable extension configured to couple the anchored end of the cable segment with the arming mechanism, the cable extension having a length selected to permit positioning of the injection device in a variety of orientations relative to, and at varying distances from, the arming mechanism, so as to facilitate discharge of the injection device without disengaging it from the arming mechanism.

20. The system of claim 18, further comprising a receiver adapted to receive a housing portion of the injection device and thereby facilitate positioning of the injection device so that the anchored end extends into the arming mechanism.

21. The system of claim 20, where the arming mechanism is configured to pull the cable segment upon operation of the actuator and, upon release of the actuator, automatically release the cable segment so as to permit removal of the injection device from engagement with the arming mechanism.

22. The system of claim 21, where the arming mechanism includes a pivoting latch configured to automatically grasp and release the anchored end of the cable segment.

23. The system of claim 16, where the arming mechanism is configured so that, upon operation of the actuator, the arming mechanism automatically grasps and pulls a cable so as to cause the spring to be compressed, and where the arming mechanism is further configured to automatically release the cable upon release of the actuator, to thereby freely permit removal of the cable from the arming mechanism.

24. The system of claim 23, where the injection orifice is formed in a nozzle of the injection device, the injection device further comprising a filling adapter secured to the nozzle adjacent the injection orifice and configured to enable an external supply of injectable fluid to be fluidly coupled with the injection orifice.

25. The system of claim 24, where the filling adapter is frangibly attached to the nozzle such that the filling adapter cannot be reattached to the nozzle after being broken away from the nozzle, and where the needle free-injection system is configured to prevent delivery of an injection from the injection orifice into an injection site until the filling adapter is broken away from the nozzle.

26. The system of claim 25, where the needle-free injection system is configured to prevent delivery of an injection into an injection site until the ability of the filling adapter to enable filling of the injection device has been disabled.
